(19) United States
(12) Patent Application Publication
(10) Pub. No.: US 2008/0032406 A1
(43) Pub. Date: Feb. 7, 2008

(54) Methods For Determining The Origin Of Sucralose Products

(75) Inventor: John C. Fry, Horsham (GB)

Correspondence Address: SNELL & WILMER L.L.P. (Main), 400 EAST VAN BUREN, ONE ARIZONA CENTER, PHOENIX, AZ, 85004-2202, US

(73) Assignee: HEALTHY BRANDS, LLC, Deerfield Beach, FL (US)

(21) Appl. No.: 11/572,782

(22) PCT Filed: Jul. 22, 2005
(86) PCT No.: PCT/US05/26040

§ 371 Date: Jan. 26, 2007

(30) Foreign Application Priority Data
Jul. 28, 2004 (GB) 0416799.5

(51) Int. Cl. G01N 33/02

(52) U.S. Cl. 436/20

(57) ABSTRACT

A method is provided for determining the origin of a sample of the artificial sweetener sucralose. Stable chlorine isotope analysis is used in the determination. Sucralose is sold under the trademark Splenda® by authorised manufacturers. The ability to determine the origin of a sample of sucralose is particularly useful to manufacturers authorised to use the Splenda® trademark, since it will enable them to monitor the extent of the unauthorised use of the trademark and allow them to take appropriate action in protecting their trademark rights accordingly.

### CROSS-REFERENCE

This application claims the benefit of U.K. Patent Application No. GB0416799.5, titled “Sucralose Analysis,” filed Jul. 28, 2004, the entire disclosure of which is incorporated herein by reference for all purposes.

### FIELD OF THE INVENTION

The present disclosure relates to methods for analyzing sucralose products to determine their origin.

### BACKGROUND

Sucralose is 1,6-dichloro-1,6-dideoxy-β-D-fructofuranosyl-4-chloro-4-deoxy-α-D-galacto-pyranoside (also abbreviated to 4,1′,6′-trichloro-4,1′,6′-trideoxygalactosucrose or TGS), a high-potency artificial sweetener, commonly known by the trademark name of Splenda®. Sucralose is made from sucrose (table sugar) by selective replacement of three hydroxyl groups on the sugar molecule with three atoms of chlorine. This change renders sucralose roughly 600 times sweeter on a weight basis than the parent sucrose. It also greatly enhances the stability of the sucralose molecule to hydrolytic breakdown and renders it almost completely indigestible by man, so that it has neither energy (calorie) content nor insulin demand. Because of its excellent taste properties, zero-calorie content, stability and safety, sucralose is becoming a popular, non-nutritive sweetener for reduced-sugar, low-calorie, and diet foods and beverages.

As used herein, the term sucralose refers to pure 4,1′,6′-trichloro-4,1′,6′-trideoxygalactosucrose, free from contaminants, impurities, conversion by-products, breakdown products, etc. The term sucralose product refers to a composition including primarily sucralose. For example, sucralose product may refer to pure sucralose or commercially pure sucralose that includes sucralose and other ingredients that are difficult to separate from the sucralose during manufacture of the sucralose. Such impurities may include sucralose breakdown products, conversion by-products, or unseparated process reagents. As used herein, sucralose generally refers to the theoretically obtainable pure sucralose while sucralose product refers to both pure sucralose and the composition that is economically obtainable when trying to produce pure sucralose. Sucralose product may be incorporated in sweetener compositions including other components, such as other sweeteners or diluents.

Additionally, as used herein, the term compounded food refers to any ingestible food or beverage made of two or more ingredients, one of which is a sucralose product. Compounded food includes sweetener compositions and manufacturing intermediates that may be used as an ingredient in the preparation of a compounded food.

Various methods of manufacturing sucralose have been developed. While the more recently developed synthetic routes are still subject to patents, the earlier synthetic routes are either off patent or are coming off patent in the near future. With these earlier methods coming off patent, doors are opening for other manufacturers, referred to as generic manufacturers, to enter the market using the synthetic routes described by the earlier patents. However, the more recently developed synthetic routes and procedures that are still protected by patents are believed to produce a higher purity sucralose product, a sucralose product with a different taste or sweetness profile, or a sucralose product through more effective or efficient routes.

Compound food manufacturers, sucralose product manufacturers, government regulators, and consumer groups need to be able to conveniently distinguish between various sources of sucralose product. One solution for establishing the origin of sucralose product is by analyzing the impurities present in the sucralose product. The current Food Chemical Codex specification for sucralose product calls for an assay of between 98 and 102%, and lays down strict, low limits on impurities such as hydrolysis products, residual methanol from the manufacturing process, and other substances related to sucralose. Despite this high purity, an analytical comparison of the impurities found in a test sample with the impurities present in a known sample may enable an analyst to determine whether the test sample came from the same source as the known sample. An analysis of relative impurities in a number of samples may theoretically allow a determination of the relationship, or lack thereof, between the various samples. This is particularly the case for commercially pure sucralose product that is supplied as an ingredient for food and beverage manufacture or for production of a retail sweetener product.

Once the sucralose product has been mixed with other ingredients to form a finished compounded food, it is much harder to analyze for impurities originating in the sucralose product. The additional difficulty in the case of compounded foods arises because sucralose product is a high-potency sweetener and only small amounts, generally less than 0.1%, are needed to sweeten foods and beverages. For example, most diet soft drinks can be fully sweetened with sucralose product alone by using about 200 mg/l (i.e. 0.02%) sucralose product in the ready-to-drink formulation. Where the food is a concentrated product, designed for dilution by the user, (for example, a powder mix from which to prepare a soft drink) sucralose product concentrations in the product as sold will be higher. For ready-to-consume, reduced-sugar products or those using blends of non-nutritive sweeteners, the amount of sucralose product will be lower, as some of the sweetness is supplied by the other sweeteners present, and the sucralose product is called upon only to supplement this sweetness and not to supply the total sweetness of the food or drink.

It will be appreciated, therefore, that when the sucralose product is incorporated into a compounded food the impurities will thus be present at a proportionately lower concentration than in commercially pure sucralose product. Thus, an impurity that might be detectable in commercially pure sucralose product could be invisible when that sucralose product is diluted a thousand fold or more by other components of the food or beverage. Moreover, these other components might themselves contribute impurities or materials which interfere with the analytical methods and/or the procedures used to isolate from the compounded food matrix the substances it is desired to analyze.

### SUMMARY

The present disclosure is directed toward methods of using stable chlorine isotope ratio analysis to produce an indicator of the origin of a sucralose product sample. The method includes providing a sample of a first sucralose product from an unknown origin and determining a stable chlorine isotope ratio (³⁷Cl:³⁵Cl) thereof. The method also includes comparing the stable chlorine isotope ratio of the first sucralose product with a stable chlorine isotope ratio for a first reference standard to produce an indicator of the origin of the first sucralose product. Methods of the present disclosure may also include comparing the origin indicator of the first sucralose product with an origin indicator for one or more sucralose products of known origin.

### DETAILED DESCRIPTION

The present description is directed to methods for determining the origin of sucralose products using stable chlorine isotope ratio analysis. The sucralose product may be present in either a substantially pure form, such as commercially available sucralose, or in the form of a compounded food product.

Stable isotopes are non-radioactive isotopes of a particular element. Only two stable isotopes of chlorine occur naturally; these are of atomic mass 35 and 37. The International Union of Pure and Applied Chemistry's Commission on Atomic Weights and Isotopic Abundances recommends that ³⁵Cl be taken as comprising 75.77% of natural chlorine while ³⁷Cl accounts for 24.23%.

As indicated previously, sucralose is produced as a result of the selective chlorination of sucrose. During the chlorination process the different chlorine isotopes (³⁵Cl or ³⁷Cl) take part in the same chemical reactions, but because the atoms of different isotopes are of different atomic masses, they react at different rates. Additionally, physical processes such as evaporation and diffusion discriminate against heavy isotopes and differences in kinetic characteristics and equilibria can result in reaction products that are isotopically heavier or lighter than their precursor materials.

This means that the isotopes of chlorine will fractionate during the manufacture of sucralose, for the reasons described above, and in a manner that depends on the synthetic route and the physical processing involved. Because physical processes involved in the manufacture bring about fractionation, the final ratio of ³⁷Cl/³⁵Cl will depend not only on the exact synthetic sequence and process, but also on factors as detailed as the size and shape of the vessels and reactors used. In addition, since the above-mentioned physical processes (that determine the stable chlorine isotope ratio) are very difficult to duplicate, they are, therefore, virtually impossible to fake. The stable chlorine isotope ratio of sucralose products is therefore likely to be unique to its place of manufacture. Moreover, because the processes, reaction times, and other factors in determining the degree of fractionation may vary between lots, or production batches, it is possible that there will be additional variations in the average stable chlorine isotope ratio of sucralose products from a particular source. Accordingly, as used herein, the methods of stable chlorine isotope ratio analysis described herein may be used to determine the origin of sucralose products, meaning the facility in which it was manufactured, and may also provide additional information regarding the manufacturing conditions of a particular lot.

The stable chlorine isotope ratio is a property of the chlorine incorporated into the sucralose product during the manufacture of the sucralose product. Therefore, detection of the ratio does not rely on some trace impurity that may or may not be present and detectable. This offers the possibility of measuring the stable isotope ratio of the sucralose product even after it has been mixed with other ingredients to form a compounded food or beverage. It is much easier to isolate a sample of sucralose product, and particularly a sample of sucralose, from such a foodstuff than minor impurities contributed by sucralose products. Sucralose products may be isolated from a compounded food in any suitable manner, some of which are described herein. Therefore, stable chlorine isotope ratio analysis provides an accurate and reliable method of determining the origin of a sucralose product, whether it be pure sucralose, a commercially available sucralose product, a sweetener comprising sucralose, or a compounded food product including sucralose.

The methods of the present disclosure may determine the origin of sucralose products through analysis of the sucralose therein. Additionally, the methods herein described may also be used to identify the origin of sucralose products through analysis of derivatives and breakdown products of sucralose. All compounds that still carry the chlorine atoms incorporated in sucralose products during manufacture are capable of being used in this method as a source of chlorine for isotopic analysis. For example, derivates of sucralose including but not limited to alkyl and aryl esters of sucralose and alkyl and aryl ethers of sucralose may be used in the methods of the present disclosure.

Furthermore, breakdown products of sucralose including, but not limited to, 1,6-dichloro-1,6-dideoxyfructose(1,6-dichlorofructose) and 4-chloro-4-deoxy-galactose(4-chlorogalactose) may be analyzed using stable chlorine isotope ratio analysis to determine the origin of the sucralose and/or the sucralose breakdown product. Likewise, derivates of breakdown products including, but not limited to, esters, ethers, oximes, hydrazones, phenylhydrazones, semicarbazones, osazones, glyconic and glycaric acids, acetals, polyhydroxy alcohols and addition compounds such as bisulphite and cyanide addition compounds are suitable. In addition, by-products of the synthesis of sucralose such as other chlorinated sugars are suitable for analysis by stable chlorine isotope ratio analysis as herein described.

It should, therefore, be understood that the term sucralose product as used herein includes sucralose and both derivatives and breakdown products of sucralose that retain one or more of the chlorine atoms incorporated therein at manufacture. The term sucralose product as used herein also includes chlorine-containing by-products of sucralose manufacture and their derivatives.

Results from the analysis of the stable chlorine isotope ratio for a sucralose product sample are expressed as a difference between the isotopic ratio of the sample and that of a reference standard. While the isotopic ratio of a sample can be measured directly, the use of a reference standard allows for calibration of the measurement equipment and greater accuracy. The difference, δ³⁷Cl, is expressed as per mil (0/00) according to Equation 1:

$${δ}^{37} Cl = 1000 * ( \frac{{R}_{Sample} - {R}_{Standard}}{{R}_{Standard}} ) \qquad Equation 1$$

Where Rₛₐₘₚₗₑ is the isotope ratio ³⁷Cl/³⁵Cl for the sucralose product sample, and R_{standard} is the isotope ratio ³⁷Cl/³⁵Cl for the reference standard.

Values for δ³⁷Cl are reported relative to the internationally-accepted stable chlorine isotope reference, Standard Mean Oceanic Chloride (SMOC) (Kaufmann et al., Nature, 309, 338-340 1984; Godon et al., Chem. Geol 207, 1-12, 2004) which has by definition, rather than by analysis, a value of δ³⁷Cl=0%o. Natural variations in δ³⁷Cl can range from −14%o to +16%o depending on the origin of the chlorine analyzed. However, for samples from the same origin, the reproducibility (standard deviation) of measurements of δ³⁷Cl is typically ±0.05%o or better when measured with a dual-inlet mass spectrometer. Accordingly, the otherwise small range of variation of δ³⁷Cl (i.e., from −14%o to +16%o) does not present a barrier to accurate discrimination between samples due to the reproducibility of the measurements for samples from a given origin.

The determination of the stable chlorine isotope ratio in the sample and the standard is typically carried out using mass spectrometric methods. This means that, prior to the determination of the stable chlorine isotope ratio for the sucralose product sample, the chlorine in the molecules of the sucralose product must be converted to a gaseous form suitable for mass spectrometric analysis. In theory, any chlorine containing gas could be used, but gaseous chlorine or hydrogen chloride may be too corrosive to many apparatus to be useful. While many other chlorine containing gases are suitable, the following description will use monochloromethane (CH₃Cl) gas as an exemplary gas for the chlorine analysis.

The generation of monochloromethane may be achieved by quantitative removal of the chlorine from molecules of the sucralose product as an inorganic chloride and subsequent generation of monochloromethane by treatment of the chloride with iodomethane. While specific exemplary methods of converting an inorganic chloride to monochloromethane are described herein, other suitable methods of converting an inorganic chloride to monochloromethane, or other chlorine-containing gas, may also be used. The chlorine in the molecules of the sucralose product is preferably liberated by alkaline hydrolysis or dehydrohalogenation, although other means such as catalytic oxidation, combustion, and reaction with lithium are feasible. Catalytic oxidation methods simultaneously convert the carbon of the sucralose product to carbon dioxide that can also be used to measure the carbon stable isotope ratio, and thus, provide further information on the origin of the sucralose, if required. However, chlorine yields from catalytic oxidation can be poorly reproducible. Without being bound by theory, it is presently believed that alkaline hydrolysis or dehydrohalogenation may result in greater reproducibility. Other methods of liberating the chlorine may be used as well.

As indicated above, alkaline hydrolysis or dehydrohalogenation is one suitable method for recovering the chlorine from the sucralose product. In one embodiment of the dehydrohalogenation process, the chlorine in the sucralose product may be recovered by heating the sucralose product with a sodium hydroxide solution at a temperature and for a time sufficient to convert the chlorine atoms into free chloride ions. The solution is then cooled and acidified with nitric acid. Excess silver nitrate is added and the chloride ions precipitated as silver chloride and quantitatively collected by filtration, such as by filtration through a glass-fibre filter paper. The chloride thus collected may be converted to chloromethane by reaction with iodomethane at elevated temperature, and subsequently purified by gas chromatography. Typical procedures for these latter steps are described by Kaufmann (Chlorine in Groundwater: Stable Isotope Distribution. PhD thesis, Tuscon (Arizona) University) 1984, Long et al, Geochim. Cosmochim Acta. 57, 2907-2912 1993 and Jendrzejewski et al. Analyt. Chem. 69, 4259-66 1997. Other methods of alkaline hydrolysis or dehydrohalogenation may be suitable for the recovery of the chlorine from the sucralose product and are within the scope of the present disclosure.

An alternative approach for recovering the chlorine from the sucralose product is catalytic oxidation. In one embodiment of the catalytic oxidation, the sucralose product may be heated with copper(II) oxide in a sealed tube to form, among other products, carbon dioxide and copper(I and II) chlorides. The carbon dioxide is then removed and retained, if required, for carbon stable isotope ratio analysis. Excess copper oxide is then removed and the copper chloride purified via precipitation as silver chloride before the conversion of the silver chloride to chloromethane. The principles of procedures for carrying out these steps are described by Holt et al, Analyt. Chem. 69, 2727-33 1997 and Jendrzejewski et al, Analyt. Chem. 69,4259-66 1997. While the principles of catalytic oxidation have been used in stable isotope ratio analysis of chlorine in other industries, such principles have not previously been applied to sucralose products. The theoretical advantage of this method is that both carbon and chlorine are recovered for stable isotope ratio analysis. However, while the method recovers carbon quantitatively, chlorine yields can be low and variable.

Another alternative method for recovering the chlorine from the sucralose products via an oxidative preparation is to combust the sucralose product in an analytical bomb using an atmosphere of high-pressure oxygen in the presence of a calcium carbonate solution to absorb combustion products. This method recovers chlorine but not carbon dioxide, and may be a little less reliable than other methods due to low recovery rates.

Yet another alternative procedure for recovering the chlorine from the sucralose product is to react the sucralose product with lithium metal at elevated temperature in a sealed tube to capture the chlorine as lithium chloride. The latter is water soluble and the chloride can be recovered subsequently by precipitation as silver chloride. However, the practical handling of lithium, which reacts with both air and water, presents some difficulties.

When the chlorine-containing gas from the sucralose product sample is monochloromethane, the reference standard gas may be a commercial sample of monochloromethane. Alternatively, the reference standard gas may be a laboratory prepared sample of monochloromethane. Such a sample may be prepared by precipitation of inorganic chloride as silver chloride and subsequent conversion of silver chloride to monochloromethane as outlined in Examples 1 and 2 below. When other suitable chlorine-containing gases are produced from the sucralose product sample, other reference standard gases may be appropriate. The stable chlorine isotope ratio of the reference standard gas may be calibrated against the stable chlorine isotope ratio of Standard Mean Oceanic Chloride (SMOC) by the industrial supplier of the reference standard gas. Alternatively, the reference standard gas may be a laboratory-prepared sample derived from SMOC or another source previously-calibrated against SMOC. The reference standard gas may be calibrated against SMOC or another source previously-calibrated against SMOC according to any suitable method.

In principle, any suitable procedure for obtaining a mass spectrum of the chlorine in the sucralose product to be compared with that of a reference standard may be implemented in cooperation with the methods of the present disclosure. One illustrative approach is to generate gaseous monochloromethane from the sucralose product and to introduce the gas into a dual inlet mass spectrometer. While dual inlet mass spectrometers are currently believed to provide the most precise measurements for determining the stable isotope ratio of the chlorine in the sucralose product sample, other techniques are feasible. For example, continuous flow mass spectrometry lends itself to on-line sample preparation, smaller sample size, faster analysis and the possibility of interfacing with other separation techniques like gas chromatography, but its precision is lower. In addition, other techniques such as thermal ionization mass spectrometry (TIMS) can also be used. This technique uses a filament coated with an inorganic chloride, such as calcium chloride, made from the chlorine source under analysis. Heating the filament generates dicaesium chloride ions (Cs₂Cl⁺) within a mass spectrometer.

As described above, many techniques may be implemented to liberate the chlorine from the sucralose product. While the techniques may be configured or implemented with the objective of recovering all the chlorine from the sucralose product, this is not always necessary. The chlorine isotopic composition is not necessarily the same at each site on the sucralose molecule. Different sites may exhibit different degrees of isotopic fractionation depending on the manufacturing process used in the production of sucralose. Accordingly, in some applications of the methods described herein it may be desirable to release the chlorine in high yield (90-100%) and preferably quantitatively to ensure that the overall isotopic ratio of the molecule is correctly represented in subsequent mass spectrometric analysis.

As previously mentioned, the techniques herein described are equally applicable to the breakdown products of sucralose, their derivatives, and to other chlorinated by-products of sucralose manufacture. Such materials, whether accidentally or deliberately produced, can be used to give information on the isotopic composition of chlorine at specific sites on their parent molecules, and thus, enhance the detail of the characterization of the chlorine content of sucralose. For example, 4-chloro-4-deoxy-galactose(4-chlorogalactose), obtainable from sucralose by controlled hydrolysis, could be employed to give information on the stable chlorine isotope ratio specifically at the carbon 4 site on the parent sucralose molecule. Likewise, the chloride released in forming 3′:6′ anhydro-sucralose as well as the chlorine on the 3′:640 anhydro-sucralose itself, both obtainable by controlled alkaline hydrolysis of sucralose, could be used to give site-specific isotopic information on chlorine.

The techniques described thus far utilize mass spectrometry and stable chlorine isotope ratio methods to determine an origin indicator for a given sucralose product sample of unknown origin, such as by Equation 1. Accordingly, in some aspects of the present disclosure, an origin indicator for a sucralose product may be determined by providing a sample of a sucralose product; converting the chlorine in the sucralose product into a gaseous form suitable for mass spectrometric analysis; determining the stable chlorine isotope ratio (³⁷Cl/³⁵Cl) of the converted chlorine in the sucralose product sample; and comparing the stable chlorine isotope ratio (³⁷Cl/³⁵Cl) as determined with the ³⁷Cl/³⁵Cl ratio for a reference standard to thereby produce an indicator of the origin of the sucralose.

The sucralose product sample may be provided in the form of substantially pure sucralose, in the form of commercially pure sucralose product, or in the form of a compounded food product. If the sucralose product is provided in the form of a compounded food product, the sucralose product must first be isolated prior to conversion of the chlorine to a gaseous-form suitable for mass spectrometric analysis. Sucralose products can be isolated from compounded foods and beverages by a variety of techniques including extraction or dissolution of solid samples into water or an alcohol, such as methanol, followed by clean-up of the solution with solid-phase extractants, such as a Sep-Pak C18 cartridge followed by a Sep-Pak Alumina N cartridge. Alternatively, preparative-scale high-performance liquid chromatography can be used to isolate sucralose product from aqueous, alcoholic, or mixed water/organic solvent solutions. Relatively simple materials such as soft drinks can be chromatographed directly or after adjustment of pH. Finally, it is not necessary to crystallize the extracted sucralose product before analysis, as a sample in solution can be hydrolysed directly.

The chlorine in the isolated sucralose product sample thus provided is then converted to a gaseous form suitable for mass spectrometric analysis. As indicated previously, a variety of different techniques can be used to obtain suitable chlorinated gaseous forms. These include catalytic oxidation, alkaline hydrolysis, dehydrohalogenation, combustion, and reaction with lithium metal, as described above.

The chlorinated gas produced from the sucralose product sample is then introduced into a mass spectrometer to determine the stable isotope ratio of chlorine in the gaseous sample. A sample of a reference standard is then introduced into the mass spectrometer to determine the stable isotope ratio of chlorine in the standard. The stable isotope ratio of the sample is then compared with that of the standard, such as by using the formula of Equation 1, to provide a difference value, or delta value, indicative of the origin of the particular sucralose product sample. In implementations incorporating a dual-inlet mass spectrometer, the chlorine-containing gas from the sucralose product sample may be introduced into a dual inlet mass spectrometer alternately with gas produced from the reference standard.

Although one or more of the preferred methods described above can produce valid results with as little as 300 micrograms of chlorine, it is generally easier to work with larger amounts, on the order of 1-3 milligrams or more. The chlorine content of pure sucralose is 26.75%, thus to have 3 mg of chlorine it is necessary to have approximately 11 mg of sucralose for analysis. A particular advantage of the methods of the present disclosure is that it is feasible to isolate such an amount from compounded foods and beverages where sucralose is an ingredient. For example, only 55 ml of a diet soft drink sweetened solely with sucralose at a concentration of 200 mg/l would provide about 11 mg of sucralose.

While the methods described above will produce an origin indicator for a given sample of sucralose product, the delta value (δ³⁷Cl) produced by Equation 1 does not alone identify the origin of the sucralose product. However, the origin indicator may be used in some implementations to determine the origin of the sucralose product, which, depending on the manufacturing conditions of the sucralose product, may enable identification of the geographic origin or the geographic origin and lot of the manufacture.

The origin of the sucralose product may be identified in a number of ways once the origin indicator is determined. For example, the origin indicator of the sucralose product sample of unknown origin may be compared to an index, table, graph, or other representation of origin indicators for sucralose products of known origin. For example, it will be possible for manufacturers of sucralose to use the present methods to determine a finger-print value, or origin indicator, for the sucralose product produced from each of their plants and, possibly, for sucralose products produced in different lots or under different manufacturing conditions at their plants. The origin indicators determined by the manufacturers may be made available in reference materials, such as indices, tables, etc. Such reference materials may be prepared by others in a similar manner.

Additionally or alternatively, the origin indicator of the sucralose product sample of unknown origin may be compared with analytically determined origin indicators for one or more sucralose product samples of known origin. For example, the sucralose product sample of unknown origin may be considered to be a first sucralose product sample, the stable chlorine isotope ratio thereof may be determined, and the origin indicator thereof may be determined according to Equation 1. Additionally, a sample of a second sucralose product of known origin may be provided, the stable chlorine isotope ratio thereof may be determined, and the origin indicator thereof may be determined according to Equation 1. Finally, the origin indicator of the first sucralose product sample and the origin indicator of the second sucralose product sample may be compared to determine whether the first sucralose product sample is of the same origin as the second sucralose product sample.

When determining the chlorine isotope ratios and the origin indicators for the first and second sucralose product samples, the analysis may be performed according to any of the methods described above. The reference standard used in the analysis of the first sucralose product sample and the reference standard used in the analysis of the second sucralose may be of substantially the same composition or may be of different compositions. In embodiments using a dual-inlet mass spectrometer, the reference standards may be from the same supply stock and therefore of similar, if not identical, compositions.

As suggested above, the origin indicator for the first sucralose product sample of unknown origin may additionally be compared to origin indicators for multiple known samples of sucralose product each having a different known origin. In such implementations of the present methods, each different known sample of sucralose product may follow the processes described herein to determine the respective stable chlorine isotope ratios and origin indicators. The origin indicator of the first sucralose product may then be compared to the origin indicators of one or more of the known samples of sucralose product of known origin to determine the origin of the first sucralose product.

It is within the scope of the present disclosure that the above described analytical methods for determining origin indicators for one or more additional samples of sucralose product of known origins may be implemented in a number of manners. For example, the methods may be implemented by manufacturers, consumers, or others to prepare an origin/origin indicator matrix, index, chart, or other reference resource. The reference resource may become a resource for a particular series of analyses over a short time period or may become a long-term resource for reference by one or more analysts when the need may arise, such as described above with reference to manufacturers preparing reference tables for their own products. Likewise, an intending purchaser of sucralose may be able to use such reference materials or similar materials to verify that deliveries originate from the same origin as pre-purchase samples and that either or both are correctly described.

Additionally or alternatively, the methods described herein may be implemented such that one or more additional samples of sucralose product of known origin may be analyzed for determination of an origin indicator, which indicator may be compared directly to one or more origin indicators for one or more samples of unknown or uncertain origin. It is within the scope of the present disclosure that the sucralose product sample of unknown origin may be analyzed first followed by analysis of one or more additional samples of known origin. In such implementations, additional samples may be analyzed until the origin of the first sample of sucralose product is determined.

In order to determine whether a sample of unknown origin is from the same origin as a known sucralose product, it is necessary to know the delta value (δ³⁷Cl), or origin indicator, of the known sucralose product. The degree of similarity in delta values that would be taken to indicate a common origin depends on the intra-lot as well as the lot-to-lot variation in delta values for sucralose products from different origins. These variations are usually characterized in terms of standard deviation for values distributed in a Gaussian fashion (or in some other distribution which can be mathematically converted to a Gaussian distribution). In terms of standard deviation, the degree of overlap required to indicate identity of origin with some quantifiable degree of confidence can be established using standard statistical techniques. Where there is no information on the spread of delta values for a particular origin, an indication of the degree of difference between two samples can be obtained by comparing the difference in the delta values of those samples with the variation in delta values obtained from repeated determinations made on the same samples. When measured as described, the standard deviation of delta values for a single, homogeneous sample is typically 0.05%o or less.

It will be appreciated that the methods of the present disclosure can be readily used for the analysis of typical food grade products. The method therefore provides a simple and convenient way of determining an origin indicator for, and thereby the origin of, a sample of sucralose product. It will, therefore, be appreciated that the methods of the present disclosure, including the methods of stable chlorine isotope ratio analysis described above, can be suitably used for determining the origin of a sample of sucralose product, either in its pure form as sucralose, in its commercially pure form as sucralose product, or in the form of a compounded food product.

The use of such a method allows authorized manufacturers or consumers of sucralose products to monitor the origin of the sucralose product in other sucralose-containing products appearing on the market. For manufacturers, such information may help them to police their trademarks and/or their patents. Additionally, such information may help them in their marketing, pricing, and distribution strategies. For consumers of sucralose products, such as manufacturers of compounded foods incorporating sucralose products, the ability to monitor the origin of the sucralose product they are using in their processes may enable them to ensure they are receiving what they purchased and may enable them to appropriately adjust their processes in the event the sucralose product origin changes. Moreover, such information regarding the origin of the sucralose product may also affect the marketing, purchasing, pricing, and other business decisions and strategies of the consumer. Similarly, regulators and other interested observers of the food products industry will be able to use the methods of the present disclosure or the results thereof in many applications.

The following examples describe selected aspects and embodiments of the present disclosure. These examples are included for illustration and are not intended to limit or define the entire scope of the present disclosure. Variations on these examples falling within the scope of the invention will be apparent to a person skilled in the art.

### EXAMPLE 1

Conversion of the Sucralose Chlorine to a Mass Spectrometric Suitable Form.

Samples of 4 to 9 mg of sucralose product (containing approximately 1.1 to 2.4 mg chlorine) were weighed with a Sartorius BP211 D semi-microbalance readable to ±0.01 mg, dissolved in 20 ml of 2% sodium hydroxide solution and heated at 100° C. for 30 minutes. The solution was then acidified to about pH 2 with nitric acid and excess 0.2M silver nitrate was added to precipitate silver chloride. The silver chloride was recovered by filtration through a glass fibre pad (Whatman GF/F 0.7 micrometer). The filter pad was dried overnight at 75° C. and inserted in a 12 mm external diameter borosilicate glass tube sealed at one end. 50 microlitres of methyl iodide (CH₃I) were added to the pad and the tube sealed under vacuum. The tube contents were allowed to react for at least 48 hours at 70-80° C. in the dark to produce monochloromethane.

The monochloromethane was separated from residual methyl iodide by purification through dual gas chromatographic columns of external diameter of ⅛ inch and a length of 2 meters filled with Porapak Q (150-180 micrometer particle size). The carrier gas was high-purity helium (99.9995% pure) at 2.4 bars pressure. The column temperature was 130° C. A thermal conductivity detector was used to detect the chloromethane and iodomethane emerging from the columns. Pure chloromethane was isolated from the column effluent by switching the excess iodomethane to waste and trapping chloromethane with liquid nitrogen. The yield of chloromethane was subsequently determined by measurement of the pressure of an accurately-known volume.

### EXAMPLE 2

Mass Spectrometric Analysis

Pure chloromethane gas from the samples prepared as outlined in Example 1 was introduced into a dual-inlet Thermo-Finnigan delta plus XP isotope ratio mass spectrometer alternately with a chloromethane reference standard. The reference gas was prepared by precipitation of inorganic chloride as silver chloride as described in Example 1 from a sample of seawater calibrated against Standard Mean Oceanic Chloride (SMOC).

δ³⁷Cl was determined according to Equation 1 by measuring the ratio of ions of mass to charge ratio (m/z) 52 (CH₃³⁷Cl⁺) to those of mass to charge ratio 50 (CH₃³⁵Cl⁺ and CH³⁷Cl⁺) and substitution of the values in Equation 1 where: Rₛₐₘₚₗₑ=(abundance of ions of m/z 52)/(abundance of ions of m/z 50) for the sample and R_{standard}=(abundance of ions of m/z 52)/(abundance of ions of m/z 50) for the reference material.

While the ions of m/z 50 include CH³⁷Cl⁺, this ion is present in negligibly small amounts so that the signal for m/z 50 is virtually wholly determined by the ion CH₃³⁵Cl⁺.

### EXAMPLE 3

Yield and Reproducibility

Ten samples of monochloromethane gas were produced as described in Example 1 from sucralose product samples taken from a single, homogeneous supply of sucralose product. The mean yield of chlorine was 100.7% (standard deviation 3.0%). The mean value of δ³⁷Cl was −0.015%o (standard deviation 0.02%o). The results of this example illustrate that from a single supply of sucralose product, the mean value of δ³⁷Cl has very little variation (i.e., a small standard deviation).

### EXAMPLE 4

Stable chlorine Isotope Analysis of Sucralose of Different Origins

Four samples of sucralose known to be of different origin were prepared and analyzed in duplicate as described in Examples 1 and 2. The mean results values of δ³⁷Cl were:

Sample 1: chlorine yield=103.3±5% δ³⁷Cl=−0.04±0.05%o

Sample 2: chlorine yield=103.9±5% δ³⁷Cl=−3.02±0.05%o

Sample 3: chlorine yield=99.4±5% δ³⁷Cl=−12.97±0.05%o

Sample 4: chlorine yield=96.8±5% δ³⁷C =−0.75±0.05%o

Example 4 applies the methods of Examples 1 and 2 to determine mean values of δ³⁷Cl for a multiple samples of sucralose product each from different origins. Example 4 illustrates that for samples of sucralose product from different origins, the values of δ³⁷Cl can vary sufficiently to provide an origin indicator as described herein.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring, nor excluding, two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower or equal in scope to the original claims, are also regarded as included within the subject matter of the inventions of the present disclosure.

Although the present invention has been shown and described with reference to the foregoing operational principles and preferred embodiments, it will be apparent to those skilled in the art that various changes in form and detail may be made without departing from the spirit and scope of the invention.

1. A method of using stable chlorine isotope ratio analysis for producing an indicator of the origin of a sucralose product sample, the method comprising:
providing a sample of a first sucralose product;
determining a stable chlorine isotope ratio (³⁷Cl:³⁵Cl) of the first sucralose product;
comparing the stable chlorine isotope ratio of the first sucralose product with a stable chlorine isotope ratio of a first reference standard to produce an origin indicator of the first sucralose product.

2. (canceled)

3. The method of claim 1, wherein the first sucralose product has been isolated from a compounded food.

4. The method of claim 3, wherein the first sucralose product has been isolated from the compounded food using liquid chromatography.

5. The method of claim 1, wherein the stable chlorine isotope ratio of the first sucralose product is determined using mass spectrometric techniques.

6. The method of claim 5, wherein chlorine atoms in the first sucralose product are converted to produce chlorine in a gaseous form suitable for mass spectrometric analysis prior to the determination of the stable chlorine isotope ratio.

7. The method of claim 6, wherein chlorine atoms in the first sucralose product is converted into a gaseous form suitable for mass spectrometric analysis using one or more techniques selected from the group comprising alkaline hydrolysis, catalytic oxidation, combustion, and reaction with lithium metal.

8. The method of claim 6, wherein the gaseous form suitable for mass spectrometric analysis is chloromethane (CH₃Cl).

9. The method of claim 6, wherein chlorine in the first sucralose product is removed quantitatively to produce chlorine in a gaseous form.

10-11. (canceled)

12. The method of claim 5, wherein the stable chlorine isotope ratio of the first sucralose product and that of the first reference standard is determined using a dual inlet mass spectrometer, the results of said stable isotope determinations being used to calculate a difference measurement between the first sucralose product and the first reference standard to provide an origin indicator of the first sucralose product.

13. The method of claim 1, wherein the origin indicator of the first sucralose product can be determined in accordance with the following formula:
$${δ}^{37} Cl = 1000 * ( \frac{{R}_{Sample} - {R}_{Standard}}{{R}_{Standard}} )$$
wherein Rₛₐₘₚₗₑ is the stable chlorine isotope ratio for the first sucralose product sample; wherein R_{standard} is the stable chlorine isotope ratio for the reference standard; and wherein the origin indicator, δ³⁷Cl, is the difference between the stable chlorine isotope ratio of the sample and that of the standard, expressed per mil (0/00).

14-17. (canceled)

17. The method of claim 1, wherein the first sucralose product sample includes one or more molecules selected from sucralose molecules, molecules of sucralose derivatives, molecules of sucralose breakdown products, molecules of derivatives of sucralose breakdown products, molecules of chlorinated sugar by-products of sucralose manufacture, and molecules of derivatives of chlorinated sugar by-products; and wherein determining a stable chlorine isotope ratio of the first sucralose product uses chlorine from at least one of the sucralose molecules, the molecules of sucralose derivatives, the molecules of sucralose breakdown products, the molecules of derivatives of sucralose breakdown products, the molecules of chlorinated sugar by-products of sucralose manufacture, and the molecules of derivatives of chlorinated sugar by-products.

18. The method of claim 1, wherein the first sucralose product sample includes molecules having one or more chlorinated sites; and wherein determining a stable chlorine isotope ratio of the first sucralose product includes determining the stable chlorine isotope ratio at one or more specific chlorinated sites.

19. The method of claim 18, wherein the stable chlorine isotope ratio at specific sites is determined by using chlorine from at least one of sucralose molecules, molecules of sucralose derivatives, molecules of sucralose breakdown products, molecules of derivatives of sucralose breakdown products, molecules of chlorinated sugar by-products of sucralose manufacture, and molecules of derivatives of chlorinated sugar by-products.

20. The method of claim 18, wherein the stable chlorine isotope ratio at specific sites is determined by removing chlorine from select sites on molecules of the first sucralose product to form specific sucralose breakdown products and determining the stable chlorine isotope ratio of the removed chlorine.

21-25. (canceled)

26. The method of claim 28, further comprising preparing a sucralose product origin/origin indicator reference including sucralose product origins and corresponding origin indicators for one or more of the additional sucralose products of known origin prior to comparing the origin indicators.

27. A method of determining the origin of a sucralose product, the method comprising:
providing a sample of a first sucralose product from an unknown origin;
determining a stable chlorine isotope ratio (³⁷Cl:³⁵Cl) of the first sucralose product;
comparing the stable chlorine isotope ratio of the first sucralose product with a stable chlorine isotope ratio of a first reference standard to produce an origin indicator of the first sucralose product; and
comparing the origin indicator of the first sucralose product with origin indicator(s) of at least one sucralose product of known origin to determine whether the first sucralose product is of the same origin as one or more of the sucralose product(s) of known origin.

28. The method of claim 27, further comprising:
providing a known sample of at least one additional sucralose product, wherein each known sample has a known origin;
determining a stable chlorine isotope ratio of one or more of the at least one additional sucralose products;
comparing the stable chlorine isotope ratio of one or more of the at least one additional sucralose products with the stable chlorine isotope ratio for corresponding reference standard(s) to produce an origin indicator of one or more of the at least one additional sucralose products; and
comparing the origin indicator of the first sucralose product with the origin indicator of one or more of the at least one additional sucralose products to determine whether the first sucralose product is of the same origin as one or more of the at least on additional sucralose products.

29. The method of claim 28, wherein the corresponding reference standard(s) and the first reference standard are of substantially the same composition.

30. The method of claim 1, wherein the first sucralose product is of a known origin.

31. The method of claim 30, wherein the method is repeated with multiple known samples of sucralose product each having a known origin, and wherein the origin indicators for each of the known samples are used to prepare an origin/origin indicator reference material.
